SEQUENCE LISTING (21 sequences)

SEQ ID NO: 1 - protein, 280 aa, Homo sapiens
MEREMVLCLAQTIHTQEDLPRPSISAEPGTVIPLGSHVTFVCRGPVGVQTFRLERDSRSTYNDTEDVSQASPSESEARFRIDSVREGNAGLYRCIYYKPPKWSEQSDYLELLVKESSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 2 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 3 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 4 - DNA, 953 bp, Homo sapiens
ccgagacactgaacaaatacgtggtcatcattgcctacgctctggtgttcctgctgagcctgctgggcaatagcctggtcatgctggtcatcctgtacagcagagtgggcagatccgtgaccgacgtgtacctgctgaatctggccctggccgatctgctgtttgccctgacactgcctatttgggccgccagcaaagtgaacggctggatcttcggcaccttcctgtgcaaggtggtgtctctgctgaaagaagtgaacttctacagcggcatcctgctgctggcctgcatcagcgttgacagatacctggccatcgtgcacgccaccagaacactgacccagaagagacacctggtcaagttcgtgtgcctcggctgttggggcctgagcatgaatctgtctctgccattcttcctgttccggcaggcctaccatcctaacaacagcagccctgtgtgctacgaggtgctgggaaacgatacagccaagtggcggatggtgctgcggatcctgcctcacaccttcggctttatcgtgcccctgttcgtgatgctgttctgctacggcttcaccctgcggaccctgttcaaagcccacatgggccagaaacaccgggccatgagagtgatctttgccgtggtgctgatcttcctgctgtgctggctgccctacaacctggtgctgcttgccgacacactgatgcggacccaagtgatccaagagagctgcgagcggcggaacaatatcggcagagccctggatgctaccgagatcctgggcttcctgcacagctgtctgaaccccatcatctacgccttcatcggccagaacttccggcacggctttctgaagattctggccatgcacggcctggtgtccaaagagttcctggccagacacagagtgaccagctacaccagcagctccgtgaacgtgtccagcaacctctga

SEQ ID NO: 5 - DNA, 983 bp, Homo sapiens
tggatgccgctccttgtgaacccgagagcctggaaatcaacaagtacttcgtggtcattatctacgccctggtgttcctgctgagcctgctgggcaatagcctggtcatgctggtcatcctgtacagcagagtgggcagatccgtgaccgacgtgtacctgctgaatctggccctggccgatctgctgtttgccctgacactgcctatttgggccgccagcaaagtgaacggctggatcttcggcaccttcctgtgcaaggtggtgtctctgctgaaagaagtgaacttctactccggcatcctgctgctggcctgcatcagcgttgacagatacctggccatcgtgcacgccaccagaacactgacccagaagcgctacctggtcaagttcatctgcctgagcatctggggactgtccctgctgcttgctctgcctgtgctgctgtttcggagaaccgtgtacagctccaacgtgtcccctgcctgctacgaggacatgggcaacaacaccgccaactggcggatgctgctgagaatcctgcctcagagcttcggcttcatcgtgcccctgctgatcatgctgttctgctacggcttcaccctgcggaccctgttcaaagcccacatgggccagaaacaccgggccatgagagtgatctttgccgtggtgctgatcttcctgctgtgctggctgccctacaacctggtgcttctggccgacacactgatgcggacccaagtgatccaagagacatgcgagcggcggaaccacatcgacagagccctggatgccacagagatcctgggaatcctgcacagctgcctgaatcctctgatctacgccttcatcggacagaagttccggcacggcctgctgaagattctggccatccacggcctgatcagcaaggacagcctgcctaaggacagcagacctagctttgtgggcagcagcagcggccacacaagcaccacactttga

SEQ ID NO: 6 - DNA, 120 bp, Homo sapiens
cgtgccaagcgaggtaagcctatccctaaccctctcctcggtctcgattctacgggatccggagccacgaacttctctctgttaaagcaagcaggagacgtggaagaaaaccccggtcct

SEQ ID NO: 7 - DNA, 2297 bp, Homo sapiens
ccatggcaagaccccacccctggtggctgtgtgtgctgggcacactggtcggactgagcgccacccctgcccctaagagctgccccgagagacactactgggctcagggcaagctgtgctgccagatgtgcgagcccggcaccttcctggtgaaagactgcgaccagcaccggaaggccgcccagtgcgatccttgcatccccggcgtgtccttcagccccgaccaccacaccagaccccactgcgagagctgccggcattgcaactctggcctgctggtccgcaactgcaccatcaccgccaacgccgagtgcgcctgcagaaacggctggcagtgccgggacaaagaatgcaccgagtgcgaccctctgcccaaccccagcctgaccgccagaagcagccaggctctgagccctcaccctcagcccacccatctgccctacgtgtccgagatgctggaagcccggacagccggccacatgcagaccctggccgacttcagacagctgcccgccagaaccctgagcacccactggcctccccagcggagcctgtgcagcagcgacttcatccggatcctggtgatcttcagcggcatgttcctggtgttcaccctggctggcgccctgttcctgcacaagcggggcagaaagaagctgctgtacatcttcaagcagcccttcatgcggcccgtgcagaccacccaggaagaggacggctgcagctgccggttccccgaggaagaggaaggcggctgcgagctgagagtgaagttcagcagaagcgccgacgcccctgcctaccagcagggccagaaccagctgtacaacgagctgaacctgggcagacgggaagagtacgacgtgctggacaagcggagaggccgggaccctgagatgggcggcaagccccagaggcggaagaaccctcaggaaggcctgtataacgaactgcagaaagacaagatggccgaggcctacagcgagatcggcatgaagggcgagcggcggagaggcaagggccacgatggcctgtaccagggcctgagcaccgccaccaaggacacctacgacgccctgcacatgcaggctctgcctccaagattaattaaccgtgccaagcgaggtaagcctatccctaaccctctcctcggtctcgattctacgggatccggagccacgaacttctctctgttaaagcaagcaggagacgtggaagaaaaccccggtcctttcgaaatgagcaacatcacggacccgcagatgtgggacttcgacgacctgaacttcaccggcatgcctccagccgacgaggattacagcccttgcatgctggaaaccgagacactgaacaaatacgtggtcatcattgcctacgctctggtgttcctgctgagcctgctgggcaatagcctggtcatgctggtcatcctgtacagcagagtgggcagatccgtgaccgacgtgtacctgctgaatctggccctggccgatctgctgtttgccctgacactgcctatttgggccgccagcaaagtgaacggctggatcttcggcaccttcctgtgcaaggtggtgtctctgctgaaagaagtgaacttctacagcggcatcctgctgctggcctgcatcagcgttgacagatacctggccatcgtgcacgccaccagaacactgacccagaagagacacctggtcaagttcgtgtgcctcggctgttggggcctgagcatgaatctgtctctgccattcttcctgttccggcaggcctaccatcctaacaacagcagccctgtgtgctacgaggtgctgggaaacgatacagccaagtggcggatggtgctgcggatcctgcctcacaccttcggctttatcgtgcccctgttcgtgatgctgttctgctacggcttcaccctgcggaccctgttcaaagcccacatgggccagaaacaccgggccatgagagtgatctttgccgtggtgctgatcttcctgctgtgctggctgccctacaacctggtgctgcttgccgacacactgatgcggacccaagtgatccaagagagctgcgagcggcggaacaatatcggcagagccctggatgctaccgagatcctgggcttcctgcacagctgtctgaaccccatcatctacgccttcatcggccagaacttccggcacggctttctgaagattctggccatgcacggcctggtgtccaaagagttcctggccagacacagagtgaccagctacaccagcagctccgtgaacgtgtccagcaacctctgagtcgac

SEQ ID NO: 8 - DNA, 2327 bp, Homo sapiens
ccatggcaagaccccacccctggtggctgtgtgtgctgggcacactggtcggactgagcgccacccctgcccctaagagctgccccgagagacactactgggctcagggcaagctgtgctgccagatgtgcgagcccggcaccttcctggtgaaagactgcgaccagcaccggaaggccgcccagtgcgatccttgcatccccggcgtgtccttcagccccgaccaccacaccagaccccactgcgagagctgccggcattgcaactctggcctgctggtccgcaactgcaccatcaccgccaacgccgagtgcgcctgcagaaacggctggcagtgccgggacaaagaatgcaccgagtgcgaccctctgcccaaccccagcctgaccgccagaagcagccaggctctgagccctcaccctcagcccacccatctgccctacgtgtccgagatgctggaagcccggacagccggccacatgcagaccctggccgacttcagacagctgcccgccagaaccctgagcacccactggcctccccagcggagcctgtgcagcagcgacttcatccggatcctggtgatcttcagcggcatgttcctggtgttcaccctggctggcgccctgttcctgcacaagcggggcagaaagaagctgctgtacatcttcaagcagcccttcatgcggcccgtgcagaccacccaggaagaggacggctgcagctgccggttccccgaggaagaggaaggcggctgcgagctgagagtgaagttcagcagaagcgccgacgcccctgcctaccagcagggccagaaccagctgtacaacgagctgaacctgggcagacgggaagagtacgacgtgctggacaagcggagaggccgggaccctgagatgggcggcaagccccagaggcggaagaaccctcaggaaggcctgtataacgaactgcagaaagacaagatggccgaggcctacagcgagatcggcatgaagggcgagcggcggagaggcaagggccacgatggcctgtaccagggcctgagcaccgccaccaaggacacctacgacgccctgcacatgcaggctctgcctccaagattaattaaccgtgccaagcgaggtaagcctatccctaaccctctcctcggtctcgattctacgggatccggagccacgaacttctctctgttaaagcaagcaggagacgtggaagaaaaccccggtcctttcgaaatggaagatttcaacatggaaagcgacagcttcgaggacttctggaaaggcgaggacctgagcaactacagctacagctctaccctgcctcctttcctgctggatgccgctccttgtgaacccgagagcctggaaatcaacaagtacttcgtggtcattatctacgccctggtgttcctgctgagcctgctgggcaatagcctggtcatgctggtcatcctgtacagcagagtgggcagatccgtgaccgacgtgtacctgctgaatctggccctggccgatctgctgtttgccctgacactgcctatttgggccgccagcaaagtgaacggctggatcttcggcaccttcctgtgcaaggtggtgtctctgctgaaagaagtgaacttctactccggcatcctgctgctggcctgcatcagcgttgacagatacctggccatcgtgcacgccaccagaacactgacccagaagcgctacctggtcaagttcatctgcctgagcatctggggactgtccctgctgcttgctctgcctgtgctgctgtttcggagaaccgtgtacagctccaacgtgtcccctgcctgctacgaggacatgggcaacaacaccgccaactggcggatgctgctgagaatcctgcctcagagcttcggcttcatcgtgcccctgctgatcatgctgttctgctacggcttcaccctgcggaccctgttcaaagcccacatgggccagaaacaccgggccatgagagtgatctttgccgtggtgctgatcttcctgctgtgctggctgccctacaacctggtgcttctggccgacacactgatgcggacccaagtgatccaagagacatgcgagcggcggaaccacatcgacagagccctggatgccacagagatcctgggaatcctgcacagctgcctgaatcctctgatctacgccttcatcggacagaagttccggcacggcctgctgaagattctggccatccacggcctgatcagcaaggacagcctgcctaaggacagcagacctagctttgtgggcagcagcagcggccacacaagcaccacactttgagtcgac

SEQ ID NO: 9 - DNA, 1100 bp, Homo sapiens
tggcaagaccccacccctggtggctgtgtgtgctgggcacactggtcggactgagcgccacccctgcccctaagagctgccccgagagacactactgggctcagggcaagctgtgctgccagatgtgcgagcccggcaccttcctggtgaaagactgcgaccagcaccggaaggccgcccagtgcgatccttgcatccccggcgtgtccttcagccccgaccaccacaccagaccccactgcgagagctgccggcattgcaactctggcctgctggtccgcaactgcaccatcaccgccaacgccgagtgcgcctgcagaaacggctggcagtgccgggacaaagaatgcaccgagtgcgaccctctgcccaaccccagcctgaccgccagaagcagccaggctctgagccctcaccctcagcccacccatctgccctacgtgtccgagatgctggaagcccggacagccggccacatgcagaccctggccgacttcagacagctgcccgccagaaccctgagcacccactggcctccccagcggagcctgtgcagcagcgacttcatccggatcctggtgatcttcagcggcatgttcctggtgttcaccctggctggcgccctgttcctgcacaagcggggcagaaagaagctgctgtacatcttcaagcagcccttcatgcggcccgtgcagaccacccaggaagaggacggctgcagctgccggttccccgaggaagaggaaggcggctgcgagctgagagtgaagttcagcagaagcgccgacgcccctgcctaccagcagggccagaaccagctgtacaacgagctgaacctgggcagacgggaagagtacgacgtgctggacaagcggagaggccgggaccctgagatgggcggcaagccccagaggcggaagaaccctcaggaaggcctgtataacgaactgcagaaagacaagatggccgaggcctacagcgagatcggcatgaagggcgagcggcggagaggcaagggccacgatggcctgtaccagggcctgagcaccgccaccaaggacacctacgacgccctgcacatgcaggctctgcctccaaga

SEQ ID NO: 10 - protein, 7 aa, Mus Musculus
GYTFTDY

SEQ ID NO: 11 - protein, 6 aa, Mus Musculus
NPYNGN

SEQ ID NO: 12 - protein, 9 aa, Mus Musculus
SSPRYALDF

SEQ ID NO: 13 - protein, 11 aa, Mus Musculus
LASQDIRNELN

SEQ ID NO: 14 - protein, 7 aa, Mus Musculus
NGKNLQS

SEQ ID NO: 15 - protein, 9 aa, Mus Musculus
LQHYSILRT

SEQ ID NO: 16 - protein, 7 aa, Homo sapiens
GFTFNIN

SEQ ID NO: 17 - protein, 8 aa, Homo Sapiens
RSKSNNYA

SEQ ID NO: 18 - protein, 8 aa, Homo Sapiens
ELRGAMDY

SEQ ID NO: 19 - protein, 11 aa, Homo Sapiens
LASQTIGTWLA

SEQ ID NO: 20 - protein, 7 aa, Homo Sapiens
AATSLAD

SEQ ID NO: 21 - protein, 9 aa, Homo Sapiens
QQLYSTPWT

(19) United States
(12) Patent Application Publication
(10) Pub. No.: US 2025/0026826 A1
(43) Pub. Date: Jan. 23, 2025

(54) ANTI-LAIR1 ANTIBODIES FOR THE TREATMENT OF CANCER

(71) Applicant: UNIVERSITY OF FLORIDA RESEARCH FOUNDATION, INCORPORATED, Gainesville, FL (US)

(72) Inventor: Jianping Huang, Gainesville, FL (US)

(21) Appl. No.: 18/276,916

(22) PCT Filed: Feb. 16, 2022
(86) PCT No.: PCT/US22/16603

§ 371 Date: Aug. 11, 2023

Related U.S. Application Data
(60) Provisional application No. 63/150,403, filed on Feb. 17, 2021.

(51) Int. Cl. C07K 16/28; A61K 39/00; A61K 39/395; A61P 35/00

(52) U.S. Cl. CPC C07K 16/2803; A61K 39/3955; A61K 39/4611; A61K 39/4631; A61K 39/464403; A61P 35/00; C07K 2317/24

(57) ABSTRACT

The present disclosure provides a method of treating cancer, comprising administering an anti-LAIR1 antibody to a subject suffering from a cancer that is resistant to checkpoint inhibitor blockade therapy.

### CROSS-REFERENCE TO RELATED APPLICATIONS

The present application claims the benefit of priority to U.S. Provisional Application No. 63/150,403, filed Feb. 17, 2021, the disclosure of which was incorporated herein by reference in its entirety.

### BACKGROUND

Glioblastoma, also known as glioblastoma multiforme (GBM), is an aggressive, stubborn brain tumor with low odds of survival for patients. The most common length of survival following diagnosis is 12 to 15 months, with fewer than 3% to 5% of subjects surviving longer than five years. Typically, treatment involves surgery, after which chemotherapy and radiation therapy are used. In cases where treatment is initially effective, the cancer usually recurs. Cancer immunotherapy represents a promising treatment approach for malignant gliomas but is currently hampered by the limited number of ubiquitously expressed tumor antigens and the profoundly immunosuppressive tumor microenvironment.

### INCORPORATION BY REFERENCE OF INFORMATION SUBMITTED ELECTRONICALLY

This application contains, as a separate part of the disclosure, a Sequence Listing in computer-readable form (Filename: 56531_Seqlisting.txt; Size: 19,161 Bytes: Created: Feb. 16, 2022), which is incorporated by reference in its entirety.

### SUMMARY

In one aspect, described herein is a method of treating cancer. The method comprises administering an anti-LAIR1 antibody to a subject suffering from cancer that is resistant to checkpoint inhibitor therapy. In some embodiments, the checkpoint inhibitor therapy is an anti-PD-1 therapy (e.g., pidilizumab, nivolumab, or pembrolizumab). Optionally, the cancer is glioblastoma. In some embodiments, the method further comprises administering a CD70 CAR T cell therapy to the subject. Optionally, the antibody binds to an epitope of human LAIR1 within amino acids 22-165 of SEQ ID NO: 1.

### BRIEF DESCRIPTION OF THE FIGURES

FIGS. 1A-1D show that LAIR1 is highly associated with other negative immune checkpoints (NICs) in primary glioblastoma. FIG. 1A is a graph showing the results of RNA-seq of primary GBM from TCGA (n=160) that were separated based the mean value of LAIR1 and compared for the expression levels of indicated NICs. The differential significance between the LAIR1 high and low is shown in the graph. FIG. 1B show that LAIR1 is upregulated on T cells upon activation. The mouse T cells were stimulated with antiCD3/28 beads and FACS analysis was performed five days later. FIGS. 1C and 1D shows that LAIR1 expression on T cells is associated with PD-1, and all PD-1+ T cells are LAIR1+. The T cells were activated and FACS was done three days later. FIG. 1D shows that LAIR1 only expresses activated T cells, and all the CD4+ Foxp3+ T cells express LAIR1.

FIGS. 2A and 2B show that inhibition of LAIR1 with an anti-LAIR1 antibody reversed myeloid-derived suppressor cell (MDSC) mediated T cell suppression in vitro. FIG. 2A is a graph showing that inhibition of LAIR1 reversed MDSC induced T cell proliferation. Mouse splenocytes were isolated from C57BL/6 mice, labeled with proliferation dye CellTrace Violet™, and activated with CD3/CD28 beads in the presence or absence of MDSCs (bone marrow-derived, isolated by Miltenyi Biotec kit) or anti-LAIR1 antibody, respectively. Flow cytometry analysis was done five days post bead activation. FIG. 2B is a graph showing that LAIR1 inhibition reversed MDSC mediated suppression the INF-γ production of CAR T cells.

FIGS. 3A and 3B show the inhibition of LAIR1 with an anti-LAIR1 antibody-induced antitumor response in a glioblastoma animal model. FIG. 3A is a graph showing tumor growth by the treatments (i.e., IgG control, CD70 CAR T cells or anti-LAIR1 antibody) over time. FIG. 3B is a graph showing the survival of the mice in each treatment group.

FIGS. 4A-4E show that inhibition of LAIR1 with an anti-LAIR1 antibody prolonged survival in a PD-1 blockade-resistant glioblastoma mouse model and provided synergistic anti-tumor response with CAR T cell therapy. FIG. 4A provides the dosing schedule of the experiment. FIG. 4B is a graph showing that treatment with the anti-LAIR1 antibody prolonged survival of the tumor burdened mice. FIG. 4C is a graph showing that treatment with the anti-LAIR1 antibody provided a superior anti-tumor efficacy that the PD-1 blockade. FIG. 4D provides the dosing schedule of an experiment as follows: C57BL/6 mice were inoculated with KR70-Luc GBM (day 0, ˜70% CD70+), and 8 days later, the mice were confirmed tumor formation by IVIS, the TBI (5Gy) was performed and randomly divided into 4 groups (n=5˜7/group). The CD70CAR T cells were given (2×10⁷/mouse, i.v.) to the indicated groups 9 days post tumor inoculation. Two days later, the anti-LAIR1 or IgG control antibody was administered (i.p. 150 μg/mouse) to the treatment group every other day for 3 doses (Days 11, 13, and 15). FIG. 4E is a graph showing that treatment with an anti-LAIR1 antibody provided a synergistic antitumor response with CAR T cell therapy.

### DETAILED DESCRIPTION

The present invention is based, in part, on the discovery that an anti-LAIR1 antibody induces a potent anti-tumor response in a PD-1 resistant glioblastoma (GBM) animal model.

As described in the Examples, a CD70-associated negative immune checkpoint (NIC), LAIR1 (expressed by GBM tissues), was shown to be responsible for myeloid-derived suppressor cell (MDSC)-associated T cell suppression; blocking of LAIR1 released the inhibition. Importantly, a potent antitumor response was observed in a syngeneic GBM mouse model when the blockade was used alone or in combination with CD70 CAR T cells.

In one aspect, described herein is a method of treating cancer comprising administering an anti-LAIR1 antibody to a subject suffering from cancer that is resistant to checkpoint inhibitor therapy. The term “resistant to checkpoint inhibitor therapy” or “resistance to checkpoint inhibitor therapy” refers to an acquired or natural resistance of a cancer sample to a checkpoint inhibitor therapy (i.e., being nonresponsive to or having reduced or limited response to the therapeutic treatment). “Resistance to checkpoint inhibitor therapy” includes a response to the therapy that is reduced by 25% or more, for example, 30%, 40%, 50%, 60%, 70%, 80%, or more, to 2-fold, 3-fold, 4-fold, 5-fold, 10-fold, 15-fold, 20-fold or more in comparison to, e.g., the same cancer sample before the resistance is acquired or a different cancer sample that is known to have no resistance to the checkpoint inhibitor blockade therapy. Resistance can be detected by reduced tumor shrinkage in response to therapy compared to other tumors or other subjects, the increased dose required to achieve a desired biological effect (if such an effect can be achieved at all), or reduced time interval between multiple doses of checkpoint inhibitor to achieve a desired biological effect. In some aspects, a tumor which is resistant to checkpoint inhibitor therapy is one which does not respond to the therapy at all clinically.

The term “checkpoint inhibitor therapy” refers to the use of agents that inhibit immune checkpoint nucleic acids and/or proteins. Inhibition of one or more immune checkpoints can block or otherwise neutralize inhibitory signaling to thereby upregulating an immune response in order to more efficaciously treat cancer. “Checkpoint proteins are molecules on the cell surface of CD4+ and/or CD8+ T cells that fine-tune immune responses by down-modulating or inhibiting an anti-tumor immune response. Targets of immune checkpoint pathways are well-known in the art and include, but are not limited to, PD-1, CTLA-4, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). Immune checkpoint signaling pathways are reviewed in Pardoll, Nature Rev Cancer 12(4): 252-264 (2012).

Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof. Other exemplary agents include, but are not limited to, RNA interference, antisense, nucleic acid aptamers, etc. that can downregulate the expression and/or activity of immune checkpoint nucleic acids, or fragments thereof. Agents can directly block the interaction between the one or more immune checkpoints and its natural receptor(s) (e.g., antibodies) to prevent inhibitory signaling and upregulate an immune response. In some embodiments, the checkpoint inhibitor therapy is anti-PD-1 therapy. “Programmed Death-1” (PD-1), also known as cluster of differentiation 279 (CD279), refers to an immunoinhibitory receptor belonging to the CD28 family. PD-1 is expressed on previously activated T cells in vivo, and binds to two ligands, PD-L1 and PD-L2. The human PD-1 sequence can be found under GenBank Accession No. U64863. In some embodiments, the anti-PD-1 therapy is pidilizumab, nivolumab or pembrolizumab. Thus, in various aspects of the disclosure, the subject is suffering from a cancer that is resistant to anti-PD-1 therapy, including (but not limited to) resistant to pidilizumab, nivolumab, or pembrolizumab therapy.

### Subject

As used herein, a “subject” means a human or animal. Usually the animal is a vertebrate such as a primate, rodent, domestic animal or game animal. Primates include, for example, chimpanzees, cynomolgous monkeys, spider monkeys, and macaques, e.g., Rhesus. Rodents include, for example, mice, rats, woodchucks, ferrets, rabbits, and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish, and salmon. In some embodiments, the subject is a mammal, e.g., a primate, e.g., a human. The terms, “individual,” “patient” and “subject” are used interchangeably herein.

A subject can be one who has been previously diagnosed with or identified as suffering from or having a condition in need of treatment (e.g., glioblastoma or another type of cancer, among others) or suffering from one or more complications related to such a condition. Optionally, the subject may have already undergone treatment for the condition or the one or more complications related to the condition. In some cases, the prior treatment that the subject has undergone is a checkpoint inhibitor therapy (e.g., anti-PD-1 therapy, such as pidilizumab, nivolumab, or pembrolizumab). In some cases, the method comprises obtaining a biopsy from the subject and determining whether the cancer is resistant to checkpoint inhibitor therapy.

A “subject in need” of treatment for a particular condition can be a subject suffering from the condition, diagnosed as having that condition, or at risk of developing that condition (e.g., cancer). In some embodiments, the methods described herein comprise administering an effective amount of an anti-LAIR1 antibody as described herein to a subject in order to alleviate a symptom of the cancer.

Exemplary cancers include, but are not limited to, breast cancer, renal cell carcinoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission, adult acute myeloid leukemia with Inv(16)(p13.1q22), CBFB-MYH11, adult acute myeloid leukemia with t(16;16)(p13.1;q22), CBFB-MYH11, adult acute myeloid leukemia with t(8;21)(q22;q22), RUNX1-RUNX1T, adult acute myeloid leukemia with t(9;11)(p22;q23), MLLT3-MLL, adult acute promyelocytic leukemia with t(15;17)(q22;q12), PML-RARA, alkylating agent-related acute myeloid leukemia, chronic lymphocytic leukemia, richter's syndrome; waldenstrom macroglobulinemia, adult glioblastoma; adult gliosarcoma, recurrent glioblastoma, recurrent childhood rhabdomyosarcoma, recurrent Ewing sarcoma, peripheral primitive neuroectodermal tumor, recurrent neuroblastoma, recurrent osteosarcoma, colorectal cancer, MSI positive colorectal cancer, MSI negative colorectal cancer, nasopharyngeal nonkeratinizing carcinoma, recurrent nasopharyngeal undifferentiated carcinoma, cervical adenocarcinoma; cervical adenosquamous carcinoma, cervical squamous cell carcinoma, recurrent cervical carcinoma, stage IVA cervical cancer, stage IVB cervical cancer, anal canal squamous cell carcinoma, metastatic anal canal carcinoma, recurrent anal canal carcinoma, recurrent head and neck cancer, carcinoma, squamous cell of head and neck, head and neck squamous cell carcinoma (HNSCC), ovarian carcinoma, colon cancer, gastric cancer, advanced GI cancer, gastric adenocarcinoma, gastroesophageal junction adenocarcinoma, bone neoplasms, soft tissue sarcoma, bone sarcoma, thymic carcinoma, urothelial carcinoma, recurrent merkel cell carcinoma, stage III merkel cell carcinoma, stage IV merkel cell carcinoma, myelodysplastic syndrome and recurrent mycosis fungoides and Sezary syndrome. In some embodiments, the cancer is glioblastoma. In some embodiments, an anti-LAIR1 antibody described herein is administered to a subject suffering from glioblastoma.

### Efficacy

The efficacy of the treatment methods described herein can be determined by the skilled clinician. However, a treatment is considered “effective treatment” if one or more of the signs or symptoms of a condition described herein is altered in a beneficial manner, other clinically accepted symptoms are improved (or even ameliorated), or a desired response is induced. Efficacy can also be measured by a failure of an individual to worsen as assessed by hospitalization, or need for medical interventions (i.e., progression of the disease is halted). Methods of measuring these indicators are known to those of skill in the art and/or are described herein.

The term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For example, the treatment provided by the methods of the present disclosure may encompass slowing the progression of the disease.

The efficacy of a method of treating a subject for cancer may be determined by any of a number of ways. Any improvement in the subject's well-being is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or burden; (7) absence of clinically detectable disease, (8) decrease in levels of cancer markers; (9) an increased patient survival rate; and/or (10) some relief from one or more symptoms associated with the disease or condition (e.g., pain). For example, the efficacy of treatment may be determined by detecting of a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In addition, treatment efficacy also can be characterized in terms of responsiveness to other treatment (e.g., chemotherapy) as appropriate.

Response may be assessed, for example for efficacy, where the size of a tumor after treatment can be compared to the initial size and dimensions as measured by, e.g., CT, PET, mammogram, ultrasound or palpation. Responses may also be assessed by caliper measurement or pathological examination of a tumor after biopsy or surgical resection.

Assessment of hyperproliferative disorder response may be done early after the onset of anti-LAIR1 antibody treatment, e.g., after a few hours, days, weeks or preferably after a few months. A typical endpoint for response assessment is upon termination of treatment with the anti-LAIR1 antibody.

As used herein, “alleviating a symptom of the cancer” is ameliorating any condition or symptom associated with cancer. As compared with an equivalent untreated control, such reduction is by at least 5%, 10%, 20%, 40%, 50%, 60%, 80%, 90%, 95%, 99% or more as measured by any standard technique known to those skilled in the art. The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disease (e.g., cancer).

It is contemplated that the methods herein reduce tumor burden, and/or reduce metastasis in the subject, and/or reduce or prevent the recurrence of tumors once the cancer has gone into remission. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

### Antibodies

The methods described herein comprise administering an anti-leukocyte-associated immunoglobulin-like receptor 1 (LAIRl) antibody to the subject. The term “antibody” refers to an intact immunoglobulin molecule (including polyclonal, monoclonal, chimeric, humanized, and/or human versions having full length heavy and/or light chains). The antibody may be any type of antibody, i.e., immunoglobulin, known in the art. In exemplary embodiments, the antibody is an antibody of class or isotype IgA, IgD, IgE, IgG, or IgM. In exemplary embodiments, the antibody described herein comprises one or more alpha, delta, epsilon, gamma, and/or mu heavy chains. In exemplary embodiments, the antibody described herein comprises one or more kappa or light chains. In exemplary aspects, the antibody is an IgG antibody and optionally is one of the four human subclasses: IgG1, IgG2, IgG3 and IgG4. Also, the antibody in some embodiments is a monoclonal antibody. In other embodiments, the antibody is a polyclonal antibody. In some aspects, the antibody is a chimeric or a humanized antibody. The term “humanized” when used in relation to antibodies refers to antibodies having at least CDR regions from a non-human source and which are engineered to have a structure and immunological function more similar to true human antibodies than the original source antibodies. For example, humanizing can involve grafting CDRs from a non-human antibody, such as a mouse antibody, into a human antibody framework. Humanizing also can involve select amino acid substitutions to make a non-human sequence look more like a human sequence.

In some aspects, the antibody is a Humaneered™ antibody. Humaneering technology converts non-human antibodies into engineered human antibodies. Humaneered™ antibodies have high affinity, and are highly similar to human germline antibody sequences. See, e.g., Tomasevic et al., Growth Factors 32: 223-235 (2014).

The anti-LAIR1 antibody (or antigen binding fragment) preferentially binds LAIR1 (e.g., human LAIR1 set forth in SEQ ID NO: 1) over other proteins. The anti-LAIR1 antibody preferably binds LAIR1 with a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. It will be appreciated that ranges having the values above as end points is contemplated in the context of the disclosure. For example, the antibody or antigen binding fragment thereof may bind human LAIR1 of SEQ ID NO: 1 with an affinity of about 1×10⁻⁷ M to about 9×10⁻¹² M or an affinity of 1×10⁻⁹ to about 9×10⁻¹².

In some or any embodiments, the antibody (or antigen binding fragment) binds to human LAIR1 of SEQ ID NO: 1, or a naturally occurring variant thereof, with an affinity (Kd) of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10¹² M, or ranging from 1×10⁻⁹ to 1×10⁻¹⁰, or ranging from 1×10⁻¹² to about 1×10⁻¹³. Affinity is determined using a variety of techniques, examples of which include an affinity ELISA assay and a surface plasmon resonance (BIAcore) assay. In some or any embodiments, the antibody (or antigen binding fragment) binds to an extracellular fragment of human LAIR1 (e.g., amino acids 22-165 of SEQ ID NO: 1). In some or any embodiments, the antibody (or antigen binding fragment) binds to an epitope within amino acids 22-165 of SEQ ID NO: 1.

“CDR” refers to the complementarity determining region within antibody variable sequences. There are three CDRs in each of the variable regions of the heavy chain and the light chain, which are designated CDR1, CDR2 and CDR3, for each of the variable regions. The term “set of six CDRs” refers to a group of three CDRs that occur in the light chain variable region and heavy chain variable region, which are capable of binding an antigen. The exact boundaries of CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDRs are obtained by, e.g., constructing polynucleotides that encode the CDR of interest and expression in a suitable host cell. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

In some embodiments, the anti-LAIR1 antibody comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 10, CDR-H2 has the sequence given in SEQ ID NO: 11, CDR-H3 has the sequence given in SEQ ID NO: 12, CDR-L1 has the sequence given in SEQ ID NO: 13, CDR-L2 has the sequence given in SEQ ID NO: 14 and CDR-L3 has the sequence given in SEQ ID NO: 15. In various aspects, the antibody (or antigen binding fragment thereof) comprises a CDR-H1 having the sequence given in SEQ ID NO: 10 with 3, 2, or 1 amino acid substitutions therein, CDR-H2 having the sequence given in SEQ ID NO: 11 with 3, 2, or 1 amino acid substitutions therein, CDR-H3 having the sequence given in SEQ ID NO: 12 with 3, 2, or 1 amino acid substitutions therein, CDR-L1 having the sequence given in SEQ ID NO: 13 with 3, 2, or 1 amino acid substitutions therein, CDR-L2 having the sequence given in SEQ ID NO: 14 with 3, 2, or 1 amino acid substitutions therein and CDR-L3 having the sequence given in SEQ ID NO: 15 with 3, 2, or 1 amino acid substitutions therein. The anti-LAIR1 antibody, in various aspects, comprises two of the CDRs, three of the CDRs, four of the CDRs, five of the CDRs or all six of the CDRs. In some embodiments, the anti-LAIR1 antibody comprises a set of six CDRs as follows: CDR-H1 of SEQ ID NO: 10, CDR-H2 of SEQ ID NO: 11, CDR-H3 of SEQ ID NO: 12, CDR-L1 of SEQ ID NO: 13, CDR-L2 of SEQ ID NO: 14 and CDR-L3 of SEQ ID NO: 15.

In some embodiments, the anti-LAIR1 antibody comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 16, CDR-H2 has the sequence given in SEQ ID NO: 17, CDR-H3 has the sequence given in SEQ ID NO: 18, CDR-L1 has the sequence given in SEQ ID NO: 19, CDR-L2 has the sequence given in SEQ ID NO: 20 and CDR-L3 has the sequence given in SEQ ID NO: 21. In various aspects, the antibody (or antigen binding fragment thereof) comprises a CDR-H1 having the sequence given in SEQ ID NO: 16 with 3, 2, or 1 amino acid substitutions therein, CDR-H2 having the sequence given in SEQ ID NO: 17 with 3, 2, or 1 amino acid substitutions therein, CDR-H3 having the sequence given in SEQ ID NO: 18 with 3, 2, or 1 amino acid substitutions therein, CDR-L1 having the sequence given in SEQ ID NO: 19 with 3, 2, or 1 amino acid substitutions therein, CDR-L2 having the sequence given in SEQ ID NO: 20 with 3, 2, or 1 amino acid substitutions therein and CDR-L3 having the sequence given in SEQ ID NO: 21 with 3, 2, or 1 amino acid substitutions therein. The anti-LAIR1 antibody, in various aspects, comprises two of the CDRs, three of the CDRs, four of the CDRs, five of the CDRs or all six of the CDRs. In some embodiments, the anti-LAIR1 antibody comprises a set of six CDRs as follows: CDR-H1 of SEQ ID NO: 16, CDR-H2 of SEQ ID NO: 17, CDR-H3 of SEQ ID NO: 18, CDR-L1 of SEQ ID NO: 19, CDR-L2 of SEQ ID NO: 20 and CDR-L3 of SEQ ID NO: 21.

In some embodiments, the anti-LAIR1 antibody is an antibody disclosed in International Publication No. WO 2018/126259, the disclosure of which is incorporated herein by reference in its entirety.

Antigen binding fragments of anti-LAIR1 antibodies (such as those described herein) are also contemplated. The antigen binding fragment can be any part of an antibody that has at least one antigen binding site, and the antigen binding fragment may be part of a larger structure (an “antibody product”) that retains the ability of the antigen binding fragment to recognize LAIR1. For ease of reference, these antibody products that include antigen binding fragments are included in the disclosure herein of “antigen binding fragment.” Examples of antigen binding fragments, include, but are not limited to, Fab, F(ab′)₂, a monospecific or bispecific Fab₂, a trispecific Fab₃, scFv, dsFv, scFv-Fc, bispecific diabodies, trispecific triabodies, minibodies, a fragment of IgNAR (e.g., V-NAR), a fragment of hcIgG (e.g., VhH), bis-scFvs, fragments expressed by a Fab expression library, and the like. In exemplary aspects, the antigen binding fragment is a domain antibody, VhH domain, V-NAR domain, VH domain, VL domain, or the like. Antibody fragments of the disclosure, however, are not limited to these exemplary types of antibody fragments. In exemplary aspects, antigen binding fragment is a Fab fragment. In exemplary aspects, the antigen binding fragment comprises two Fab fragments. In exemplary aspects, the antigen binding fragment comprises two Fab fragments connected via a linker. In exemplary aspects, the antigen binding fragment comprises or is a minibody comprising two Fab fragments. In exemplary aspects, the antigen binding fragment comprises, or is, a minibody comprising two Fab fragments joined via a linker. Minibodies are known in the art. See, e.g., Hu et al., Cancer Res 56: 3055-3061 (1996). In exemplary aspects, the antigen binding fragment comprises or is a minibody comprising two Fab fragments joined via a linker, optionally, comprising an alkaline phosphatase domain.

A domain antibody comprises a functional binding unit of an antibody, and can correspond to the variable regions of either the heavy (V_{H}) or light (V_{L}) chains of antibodies. A domain antibody can have a molecular weight of approximately 13 kDa, or approximately one-tenth of a full antibody. Domain antibodies may be derived from full antibodies such as those described herein.

In some embodiments, the scFv is attached to a human Fc domain. In some embodiments, the Fc domain does not activate Fc effector functions.

### Methods of Antibody or Antigen Binding Fragment Production

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5th Ed., Garland Publishing, New York, NY (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 Al). Antibodies may also be produced by inducing in vivo production in the lymphocyte population or by screening recombinant immunoglobulin libraries or panels of highly specific binding reagents as disclosed in Orlandi et al (Proc Natl Acad Sci 86: 3833-3837; 1989), and Winter G and Milstein C (Nature 349: 293-299, 1991). If the full sequence of the antibody or antigen-binding fragment is known, then methods of producing recombinant proteins may be employed. See, e.g., “Protein production and purification” Nat Methods 5(2): 135-146 (2008). In some embodiments, the antibodies (or antigen binding fragments) are isolated from cell culture or a biological sample if generated in vivo.

Phage display also can be used to generate the antibody of the present disclosures. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3rd Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150). Related methods also are described in U.S. Pat. Nos. 5,403,484; 5,571,698; 5,837,500; 5,702,892. The techniques described in U.S. Pat. Nos. 5,780,279; 5,821,047; 5,824,520; 5,855,885; 5,858,657; 5,871,907; 5,969,108; 6,057,098; and 6,225,447.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 Bl, and United Kingdom Patent No. 2188638. Humanized antibodies can also be generated using the antibody resurfacing technology described in U.S. Pat. No. 5,639,641 and Pedersen et al., J. Mol. Biol, 235, 959-973 (1994). A preferred chimeric or humanized antibody has a human constant region, while the variable region, or at least a CDR, of the antibody is derived from a non-human species. Methods for humanizing non-human antibodies are well known in the art. (See, e.g., U.S. Pat. Nos. 5,585,089, and 5,693,762.)

Techniques developed for the production of “chimeric antibodies,” e.g., the splicing of mouse antibody genes to human antibody genes to obtain a molecule with appropriate antigen specificity and biological activity, can be used (Morrison et al., Proc Natl Acad Sci 81: 6851-6855 (1984); Neuberger et al., Nature 312: 604-608 (1984); Takeda et al., Nature 314: 452-454 (1985)). Alternatively, techniques described for the production of single chain antibodies (U.S. Pat. No. 4,946,778) can be adapted to produce ANGPTL4-specific single chain antibodies.

Likewise, using techniques known in the art to isolate CDRs, compositions comprising CDRs are generated. Compositions comprising one, two, and/or three CDRs of a heavy chain variable region or a light chain variable region of a monoclonal antibody can be generated. The CDRs of exemplary antibodies are provided herein as SEQ ID NOs: 4-9 and 12-17. Techniques for cloning and expressing nucleotide and polypeptide sequences are well-established in the art (see, e.g., Sambrook et al., Molecular Cloning: A Laboratory Manual, 2nd Edition, Cold Spring Harbor, New York (1989)). The amplified CDR sequences are ligated into an appropriate expression vector. The vector comprising one, two, three, four, five and/or six cloned CDRs optionally contains additional polypeptide encoding regions linked to the CDR.

Chemically constructed bispecific antibodies may be prepared by chemically cross-linking heterologous Fab or F(ab′)₂ fragments by means of chemicals such as heterobifunctional reagent succinimidyl-3-(2-pyridyldithiol)-propionate (SPDP, Pierce Chemicals, Rockford, Ill.). The Fab and F(ab′)₂ fragments can be obtained from intact antibody by digesting it with papain or pepsin, respectively (Karpovsky et al., J. Exp. Med. 160:1686-701 (1984); Titus et al., J. Immunol., 138:4018-22 (1987)).

Methods of testing antibodies for the ability to bind to an epitope of LAIR1, regardless of how the antibodies are produced, are known in the art and include, e.g., radioimmunoassay (RIA), ELISA, Western blot, immunoprecipitation, surface plasmon resonance (e.g., BIAcore), and competitive inhibition assays (see, e.g., Janeway et al., infra, and U.S. Patent Application Publication No. 2002/0197266).

A single-chain variable region fragments (scFv), which consists of a truncated Fab fragment comprising the variable (V) domain of an antibody heavy chain linked to a V domain of an antibody light chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)).

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol Eng. 2001 18:95-108, (2001) and Todorovska et al., J Immunol Methods. 248:47-66, (2001).

### Pharmaceutical Compositions

Pharmaceutical compositions comprising an anti-LAIR1 antibody or antigen binding fragment thereof described herein are also contemplated. In some embodiments, the pharmaceutical composition contains formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, methionine or lysine); antimicrobials; antioxidants (such as reducing agents, oxygen/free-radical scavengers, and chelating agents (e.g., ascorbic acid, EDTA, sodium sulfite or sodium hydrogen-sulfite)); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter-ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo, ed.), 1990, Mack Publishing Company.

Selection of the particular formulation materials described herein may be driven by, for example, the intended route of administration, delivery format and desired dosage. See, for example, REMINGTON'S PHARMACEUTICAL SCIENCES, supra. The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments, the composition may be prepared for storage by mixing the selected composition having the desired degree of purity with optional formulation agents (REMINGTON'S PHARMACEUTICAL SCIENCES, supra) in the form of a lyophilized cake or an aqueous solution. Further, in some embodiments, the antibody or (antigen binding fragment thereof) may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the composition may be provided in the form of a pyrogen-free, parenterally acceptable aqueous solution comprising the desired antibody or fragment in a pharmaceutically acceptable vehicle. A particularly suitable vehicle for parenteral injection is sterile distilled water in which the antibody or fragment is formulated as a sterile, isotonic solution, properly preserved. In certain embodiments, implantable drug delivery devices may be used to introduce the desired antibody (or antigen binding fragment thereof).

Additional pharmaceutical compositions, including formulations involving antigen binding proteins in sustained- or controlled-delivery formulations are contemplated herein. Techniques for formulating a variety of other sustained- or controlled-delivery means, such as liposome carriers, bio-erodible microparticles or porous beads and depot injections, are available in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP133988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP036676; EP088046 and EP143949, incorporated by reference.

Embodiments of the antibody formulations can further comprise one or more preservatives.

Administration of the compositions described herein will be via any route so long as the target tissue is available via that route. The pharmaceutical compositions may be introduced into the subject by any method, e.g., by intravenous, intradermal, intramuscular, intramammary, intraperitoneal, intrathecal, intraocular, retrobulbar, intrapulmonary (e.g., term release), by oral, sublingual, nasal, anal, vaginal, or transdermal delivery, or by surgical implantation at a particular site.

### Dosage

In some embodiments, one or more doses of the antibody or antigen binding fragment are administered in an amount and for a time effective to treat cancer a subject.

For example, one or more administrations of an antibody or antigen binding fragment thereof described herein are optionally carried out over a therapeutic period of, for example, about 1 week to about 24 months (e.g., about 1 month to about 12 months, about 1 month to about 18 months, about 1 month to about 9 months or about 1 month to about 6 months or about 1 month to about 3 months). In some embodiments, a subject is administered one or more doses of an antibody or fragment thereof described herein over a therapeutic period of, for example about 1 month to about 12 months (52 weeks) (e.g., about 2 months, about 3 months, about 4 months, about 5 months, about 6 months, about 7 months, about 8 months, about 9 months, about 10 months, or about 11 months).

It may be advantageous to administer multiple doses of the antibody or antigen binding fragment at a regular interval, depending on the therapeutic regimen selected for a particular subject. In some embodiments, the antibody or fragment thereof is administered periodically over a time period of one year (12 months, 52 weeks) or less (e.g., 9 months or less, 6 months or less, or 3 months or less). In this regard, the antibody or fragment thereof is administered to the human once every about 3 days, or about 7 days, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 6 months, or 12 months.

In various embodiments, one or more doses comprising from about 50 milligrams to about 1,000 milligrams of the antibody or antigen binding fragment thereof are administered to a subject (e.g., a human subject). For example, a dose can comprise at least about 5 mg, at least about 15 mg, at least about 25 mg, at least about 50 mg, at least about 60 mg, at least about 70 mg, at least about 80 mg, at least about 90 mg, at least about 100 mg, at least about 120 mg, at least about 150 mg, at least about 200 mg, at least about 210 mg, at least about 240 mg, at least about 250 mg, at least about 280 mg, at least about 300 mg, at least about 350 mg, at least about 400 mg, at least about 420 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg or up to about 1,000 mg of antibody. Ranges between any and all of these endpoints are also contemplated, e.g., about 50 mg to about 80 mg, about 70 mg to about 140 mg, about 70 mg to about 270 mg, about 75 mg to about 100 mg, about 100 mg to about 150 mg, about 140 mg to about 210 mg, or about 150 mg to about 200 mg, or about 180 mg to about 270 mg. The dose is administered at any interval, such as multiple times a week (e.g., twice or three times per week), once a week, once every two weeks, once every three weeks, or once every four weeks.

In some embodiments, the one or more doses can comprise between about 0.1 to about 50 milligrams (e.g., between about 5 and about 50 milligrams), or about 1 to about 100 milligrams, of antibody (or antigen binding fragment thereof) per kilogram of subject body weight (mg/kg). For example, the dose may comprise at least about 0.1 mg/kg, at least about 0.5 mg/kg, at least about 1 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least 12 mg/kg, at least 13 mg/kg, at least 14 mg/kg, at least about 15 mg/kg, at least 16 mg/kg, at least 17 mg/kg, at least 18 mg/kg, at least 19 mg/kg, at least about 20 mg/kg, at least 21 mg/kg, at least 22 mg/kg, at least 23 mg·kg, at least 24 mg·kg, at least about 25 mg/kg, at least about 26 mg/kg, at least about 27 mg/kg, at least about 28 mg/kg, at least about 29 mg/kg, at least about 30 mg/kg, at least about 31 mg/kg, at least about 32 mg/kg, at least about 33 mg/kg, at least about 34 mg/kg, at least about 35 mg/kg, at least about 36 mg/kg, at least about 37 mg/kg, at least about 38 mg/kg, at least about 39 mg/kg, at least about 40 mg/kg, at least about 41 mg/kg, at least about 42 mg/kg, at least about 43 mg/kg, at least about 44 mg/kg, at least about 45 mg/kg, at least about 46 mg/kg, at least about 47 mg/kg, at least about 48 mg/kg, at least about 49 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or up to about 100 mg/kg. Ranges between any and all of these endpoints are also contemplated, e.g., about 1 mg/kg to about 3 mg/kg, about 1 mg/kg to about 5 mg/kg, about 1 mg/kg to about 8 mg/kg, about 3 mg/kg to about 8 mg·kg, about 1 mg/kg to about 10 mg/kg, about 1 mg/kg to about 20 mg/kg, about 1 mg/kg to about 40 mg/kg, about 5 mg/kg to about 30 mg/kg, or about 5 mg/kg to about 20 mg/kg.

### Combination Therapy

In various embodiments, the methods described herein may comprise administering a an additional anti-cancer therapy to the subject. An anti-cancer therapy can be, e.g., CAR T cell therapy, chemotherapy, radiation therapy, chemo-radiation therapy, immunotherapy, hormone therapy, surgery or stem cell therapy.

In some embodiments, the additional anti-cancer therapy is CAR T cell therapy. The term “CAR-T” or the term “CAR T cell” refers to a genetically engineered T cell that produces a chimeric antigen receptor (CAR) on the surface of the cell. The backbone of a CAR-T or CAR T cell is a T cell.

In some situations, T cells are collected from the body of a subject or a patient via apheresis, a process that withdraws blood from the body and removes one or more blood components (such as plasma, platelets or white blood cells). The T cells collected from the body are then genetically engineered to produce a particular chimeric antigen receptor (CAR) on their surface. After the engineering, the T cells are known as chimeric antigen receptor (CAR) T cells. The CAR T cells are expanded by growing in a laboratory and then administered to the subject or patient (i.e., the recipient is the cell donor), or another subject or patient (i.e., the recipient is not the cell donor). The CAR T cells recognize and kill cancer cells that express a targeted antigen on their surface.

In some embodiments, the methods described herein comprise administering a CD70 CAR T cell therapy to the subject. CD70 CAR T cells are further described in International Publication No. WO 2019/051047, the disclosure of which is incorporated herein by reference in its entirety. In some embodiments, the CD70 CAR T cell therapy comprises administering a modified T cell to the subject, wherein the modified T cell comprises a nucleic acid encoding a recombinant protein comprising an interleukin 8 (IL-8) receptor attached to a chimeric antigen receptor (CAR) via a hinge domain, wherein the CAR comprises an antigen binding domain, a transmembrane domain, and a CD3 zeta signaling domain. In some embodiments, the antigen binding domain is an extracellular portion of CD27 (full-length amino acid sequence set forth in SEQ ID NO: 3). In some embodiments, the hinge domain is fuV5P2A (nucleotide sequence set forth in SEQ ID NO: 6). In some embodiments, the CD70 CAR T cell expresses CD70CAR-fuV5P2A-CXCR1 (nucleotide sequence set forth SEQ ID NO: 7) or CD70CAR-fuV5P2A-CXCR2 (nucleotide sequence set forth in SEQ ID NO: 8).

In some embodiments, the modified T cell co-expresses an IL-8 receptor and a CD70 CAR on the cell surface. In some embodiments, the IL-8 receptor is CXCR1 (nucleotide sequence set forth in SEQ ID NO: 4) or CXCR2 (nucleotide sequence set forth in SEQ ID NO: 5). In some embodiments, the CD70 CAR comprises a nucleotide sequence set forth in SEQ ID NO: 9.

In some embodiments, the LAIR1 antibody enhances the efficacy of the CAR T cell therapy. For example, in some embodiments, the combination of the anti-LAIR1 antibody with the CD70 CAR T cell reduces tumor burden by at least 10% (e.g., at least 15%, at least 20%, at least 25%, at least 30%, at least 35%, at least 40%, at least 50% or more) than treatment with the CD70 CAR T cell therapy alone.

In accordance with some embodiments, the subject is administered a chemotherapeutic agent in combination with the methods described herein. Exemplary chemotherapeutic agents include, but are not limited to, a platinum chemotherapeutic agent, an anthracycline therapeutic agent, or an alkylating chemotherapeutic agent. Non-limiting examples of chemotherapeutic agents include an anthracycline (e.g., doxorubicin (such as liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzumab, gemtuzumab, rituximab, tositumomab), an antimetabolite (including, e.g., folic acid antagonists, pyrimidine analogs, purine analogs and adenosine deaminase inhibitors (such as fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HC1 (Treanda®). Exemplary mTOR inhibitors include, e.g., temsirolimus; ridaforolimus (formally known as deferolimus, (lR,2R,45)-4-[(2R)-2 [(1R,95,125,15R,16E,18R,19R,21R,235,24E,26E,28Z,305,325,35R)-l,18-dihydroxy-19,30-dimethoxy-15,17,21,23, 29,35-hexamethyl-2,3,10,14,20-pentaoxo-l l,36-dioxa-4-azatricyclo[30.3.1.04′9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOl); rapamycin (AY22989, Sirolimus®); simapimod (CAS 164301-51-3); emsirolimus, (5-{2,4-Bis[(35)-3-methylmorpholin-4-yl]pyrido[2,3-(i]pyrimidin-7-yl}-2-methoxyphenyl)methanol (AZD8055); 2-Amino-8-[iraw5-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[l,4-dioxo-4-[[4-(4-oxo-8-phenyl-4H-l-benzopyran-2-yl)morpholinium-4-yl]methoxy]butyl]-L-arginylglycyl-L-a-aspartylL-serine-, inner salt (SF1126, CAS 936487-67-1), and XL765. Exemplary immunomodulators include, e.g., afutuzumab (available from Roche); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon 7, CAS 951209-71-5, available from IRX Therapeutics). Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-l-(((5)-4-methyl-l-((R)-2-methyloxiran-2-yl)-l-oxopentan-2-yl)amino)-l-oxo-3-phenylpropan-2-yl)-2-((5)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(llS′)-2-[(2R)-2-methyl-2-oxiranyl]-2-oxo-l-(phenylmethyl)ethyl]-L-serinamide (ONX-0912).

Chemotherapeutic agent for use with methods and compositions describe herein include, but are not limited to agents described in, for example, Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In accordance with some embodiments, the subject is administered radiation therapy in combination with the methods described herein. Radiation therapy, according to the invention disclosed herein, encompasses both non-invasive (external) and invasive (internal) radiation therapies. In an external radiation therapy, treatment is affected by radiation sources outside the body, whereas in an invasive radiation therapy treatment is affected by radiation sources planted inside the body.

In accordance with some embodiments, the subject is administered a chemo-radiation therapy, e.g., a combination of chemotherapy and radiation therapy, in combination with the methods described herein.

In accordance with some embodiments, the subject is administered hormone therapy in combination with the methods described herein. Hormone therapy is designed to add, block, or remove hormones from the body to, e.g., halt or slow the growth of cancer cells. Hormone therapy can include administration of, e.g., progesterone, oophorectomy, tamoxifen, gonadotropin-releasing hormone (GnRH) agonists or analogues and androgen therapy. Hormone therapy can also refer to removing glands, e.g., thyroid, pancreas, and ovary, to reduce the levels of hormones in the body. Hormone therapies are known in the art and can be administered by a skilled person.

In accordance with some embodiments, the subject is administered stem cell therapy in combination with the methods described herein. Stem cell therapy can comprise removing a subjects stem cells prior to receiving treatment to destroy all stem cells (e.g., chemotherapy, radiotherapy, or a combination thereof). Stems cells can be re-administered to the patient following such treatment (e.g., a stem cell transplant). A stem cell transplant can be autologous, or allogenic. A stem cell transplant can be a tandem transplant (e.g., two or more transplants in a row), a mini-transplant (e.g., a subject's immune system is suppressed less than a typical transplant), or a syngeneic stem cell transplant (e.g., allogeneic stem cells received from an identical twin). Cancers that can be treated with stem cell therapy include but are not limited to leukemias, lymphomas, multiple myeloma, testicular cancer, neuroblastoma, and certain childhood cancers.

### Kits

Once a pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the disclosure may each contain both a first container having a dried protein and a second container having an aqueous formulation. In certain embodiments, kits containing single and multi-chambered pre-filled syringes (e.g., liquid syringes and lyosyringes) are provided.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein.

The technology described herein is further illustrated by the following examples which in no way should be construed as being further limiting.

### EXAMPLES

### Example 1—LAIR1 is Highly Associated with Other Negative Immune Checkpoints

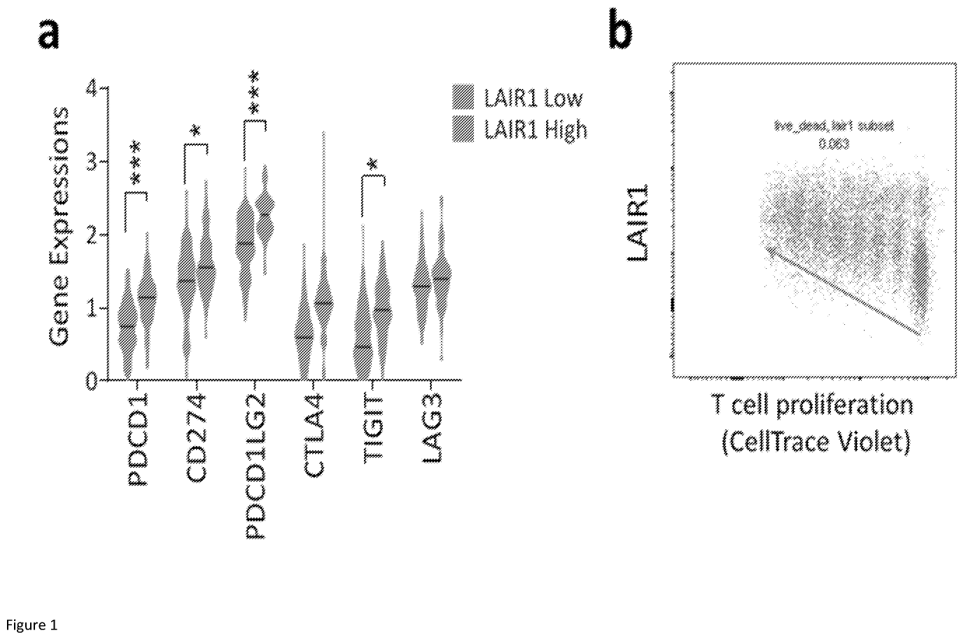

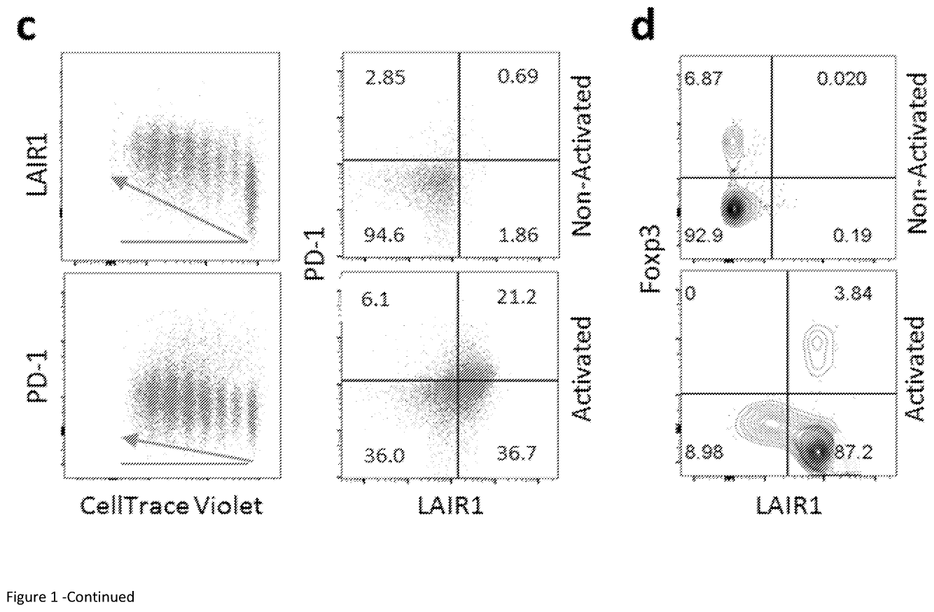

Known negative immune checkpoints (NICs), including PD-1 (PDCD1), PD-L1 (CD274), PDCD1LG2, CTLA-4, TIGIT, and LAG3 were evaluated using the RNA-seq data set in the TCGA. RNA-seq of primary GBM from TCGA (n=160) were separated based the mean value of LAIR1 and compared for the expression levels of indicated NICs. As shown in FIG. 1A, the results show that LAIR1 high tumors also express relatively higher levels of some of the NICs, including PDCD1, CD274, PDCD1LG2, and TIGIT.

Next, the LAIR1 expression on T (cells with or without activation) was evaluated. Mouse T cells were isolated from spleen (2×105) and labeled with CellTrace Violet dye, and activated with or without CD3/CD28 bead (cell:bead=4:1). The cells were analyzed by flow cytometry using anti-CD3, anti-PD-1 and anti-LAIR1 antibodies. It was determined that the activated and proliferative T cells expressed elevated LAIR1 (FIG. 1B). Interestingly, the PD-1 expression is a bit behind the LAIR1 upon the activation (FIG. 1C, and all the PD-1+ T cells after the activation are LAIR1+ (FIG. 1C). LAIR1 only expresses activated T cells, and all the CD4+ Foxp3+ T cells express LAIR1 (FIG. 1D).

### Example 2—LAIR1 Blockade Releases the T Cell Suppression Induced by MDSCs In Vitro

LAIR1 is predominantly expressed on myeloid-derived suppressor cells (MDSCs), a heterogeneous population of cells inhibiting T cell function.

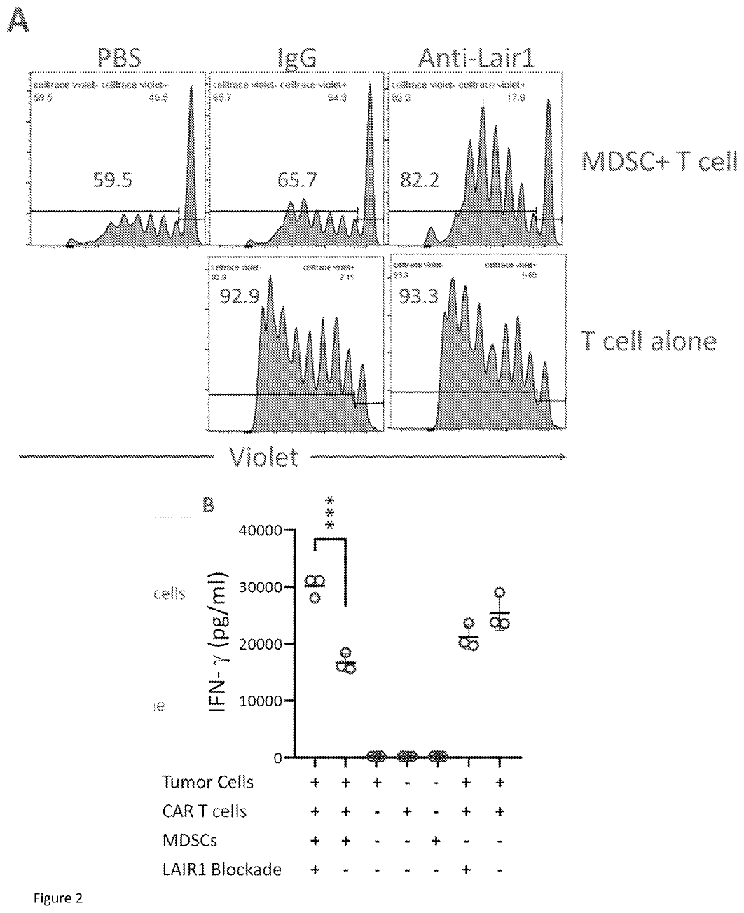

Activated T cells were co-cultured with MDSCs in the absence or presence of an anti-LAIR1 blocking antibody comprising the CDRs set forth in SEQ ID NOs: 10-15. The results show that the LAIR1 blocking antibody efficiently blocked the surface expression of LAIR1, increases the T cell proliferation that was inhibited by the MDSCs (upper panel), but it does not influence the T cell itself (lower panel) (FIG. 2A) Furthermore, the INF-γ production suppressed by the MDSCs was relieved by the LAIR1 blockade, and it did not influence the T cells directly since no significant change in the INF-γ production when the MDSCs were absent (FIG. 2B).

### Example 3—LAIR1 Blockade Induced a Potent Anti-Tumor Response in a PD-1 Resistant Glioblastoma Animal Model

Next, the impact of LAIR1 on tumor progression using a PD-1 blockade resistant GBM model (Flores et al., Nature Communications. 2018; 9(1):4313) was performed.

C57BL/6 mice were inoculated with KR70-Luc GBM (day 0, ˜70% CD70⁺), and 8 days later, the mice were confirmed tumor formation by IVIS, the TBI (5Gy) was performed and randomly divided into 4 groups (n=5˜7/group). The CD70CAR T cells (described in International Publication No. WO 2019/051047, the disclosure of which are incorporated herein by reference in its entirety) were administered (2×10⁷/mouse, i.v.) to the indicated groups 9 days post tumor inoculation. Two days later, the Armenian Hamster anti-mouse LAIR1 (comprising the CDR sequences set forth in SEQ ID NOs: 10-15) or IgG control antibody was administered (i.p. 150 μg/mouse) to the treatment group every other day for three doses (Days 11, 13 and 15). The tumor size was measured by lVIS.

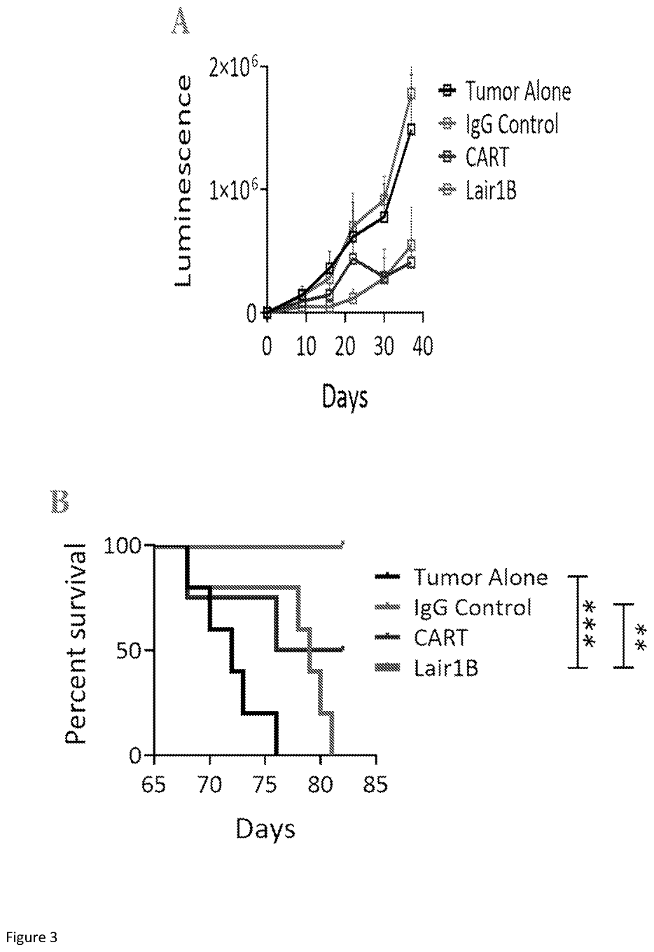

The results showed that both CAR T cells and LAIR1 blockade significantly inhibited tumor growth (FIG. 3A). As shown in FIG. 3B, treatment with the anti-LAIR1 blockade provided superior protection and delivered a better survival benefit to the treated mice than the CAR T cells.

### Example 4—Anti-Tumor Efficacy of a Humanized Anti-LAIR Antibody in an Tumor Animal Model

LAIR1 mAb will be assessed in several cancer models to characterize the ability of an anti-LAIR1 mAb to provide an antitumor response alone and in combination with CAR T cells. Two experimental models will be used:

A) Human tumor cell-derived xenograft (CDX) model: NRG mice will be used to characterize LAIR1 blockade in reversing the MDSCs-LAIR1 induced immunosuppression and enhance the CAR T cell functions. GBM patient primary tumors (pGBM #3 or pGBM #4, CD70+ clines, and overall survival between male and female mice are the same) described previously (Jin et al., Neuro Oncol. 2018 Jan. 10; 20(1):55-65; and Jin et al., Nat Commun. 2019 Sep. 5; 10(1):4016, the disclosure of which are incorporated herein by reference in their entireties), will be used for the tumor (5×10⁴) inoculation. Primary tumor cells will be co-injected with human MDSCs (5×10⁴) isolated from GBM patients' PBMC (IRB201400364) using Miltenyi Biotec isolation kit. Four groups of NRG mice (6 mice/group, both sex) will be used.

B) Patient-derived xenograft (PDX) model: Since the CDX model possesses incomplete immune components such as T lymphocytes, the Onco-Hu® platform better recapitulates human tumor and immune-cell interactions in vivo. Four groups of the Onco-Hu® mice (3 mice/group, both sex) will be used. The experimental design and outcomes for experiments A and B are shown in Table 1.

TABLE 1
Experimental Design and evaluation

| Groups | Treatment | Concentration | Schedules | Flow up |
|---|---|---|---|---|
| 1 | GBM alone | 5 × 10⁴ tumor/mouse | Day 0 | Tumor |
| | | 5 × 10⁴ MDSCs/mouse | | growth |
| | | (only for A) | | by IVIS |
| 2 | GBM + | 5 × 10⁴ tumor/mouse | Day 0 | |
| | IgG mAb | 5 × 10⁴ MDSCs/mouse | 2 days post tumor | |
| | | (only for A) | formation (PTF), and | |
| | | 150 μg/mouse mAb | 3 doses every 2 days | |
| 3 | GBM + | 5 × 10⁴ tumor/mouse | Day 0 | TME |
| | LAIR1 mAb | 5 × 10⁴ MDSCs/mouse | 2 days PTF, and 3 | analysis |
| | | (only for A) | doses every 2 days | |
| | | 150 μg/mouse mAb | | |
| 4 | GBM + | 5 × 10⁴ tumor/mouse | Day 0 | |
| | 8R-70CAR T | 5 × 10⁴ MDSCs/mouse | 2 days PTF, and a | |
| | | (only for A) | single dose CAR T | |
| | | 2 × 10⁶ cells /mouse | | |
| 5 | GBM + | 5 × 10⁴ tumor/mouse | Day 0 | Survival |
| | 8R-70CAR T + | 5 × 10⁴ MDSCs/mouse | 2 days PTF, a single | |
| | LAIR1 mAb | (only for A) | dose CAR T, and 3 | |
| | | 2 × 10⁶ cells/mouse | doses of mAb | |
| | | 150 μg/mouse mAb | | |

Several approaches will be utilized for the tumor microenvironment (TME) analysis. Single-cell suspension of tumors from the treated mice will be analyzed by flow cytometry and single-cell RNA-seq. In addition, the tumor tissue sections will also be prepared for multiplex IHC and/or GeoMx® Digital Spatial Profiler (GeoMx DSP) of the NanoString platform (www.nanostring.com). The overall landscapes (pathways and gene sets) of the different treatment groups will be assessed and compared. Also, the specific cell populations (T cells, NK cell, DC, macrophages, and MDSCs) and molecules (CD70, PD-L1 on tumors and MDSCs, PD-1 and LAIR1 on T cells) will be focused on. The differences in all these parameters will be compared among the groups.

### Example 4—LAIR1 is Overexpressed on Myeloid Cells and Increased Tumor-Bearing (TB) Mice

Flow cytometry analysis was performed on the cells of healthy and TB (with orthotopic glioma, KR-158B) mice spleen and tumor. The CD45+LAIR+ cells were measured. The LAIR1+CD45+ cells inside the tumor were determined to be CD3−. LAIR1 was determined to be expressed on the myeloid cells, not on tumor cells. Our analysis demonstrates that the LAIR1 was mainly detected on tumor-associated myeloid cells (TAMCs) inside the tumors. The cancer core was found to have more LAIR1+ cells than the tissue adjacent to the tumor. Together, the results pinpoint the primary cell populations of LAIR1+ cells in glioblastoma to the TAMCs, a pro-cancer immune cell population that composes up to 50% of the total tumor mass in glioblastoma.

### Example 5—LAIR1 Blockade Prolonged Survival in a PD-1 Blockade-Resistant GBM Mouse Model

The following Example demonstrates that blocking LAIR1 using antibody blockade prolongs survival in a PD-1 blockade-resistant GBM mouse model and provides a synergistic antitumor response with CAR T cell therapy.

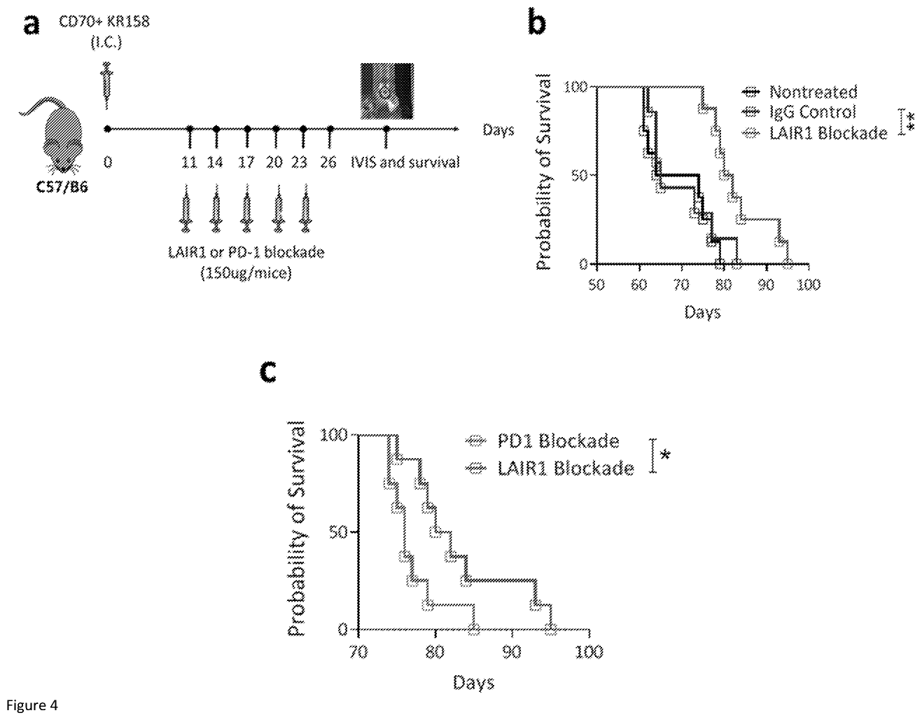

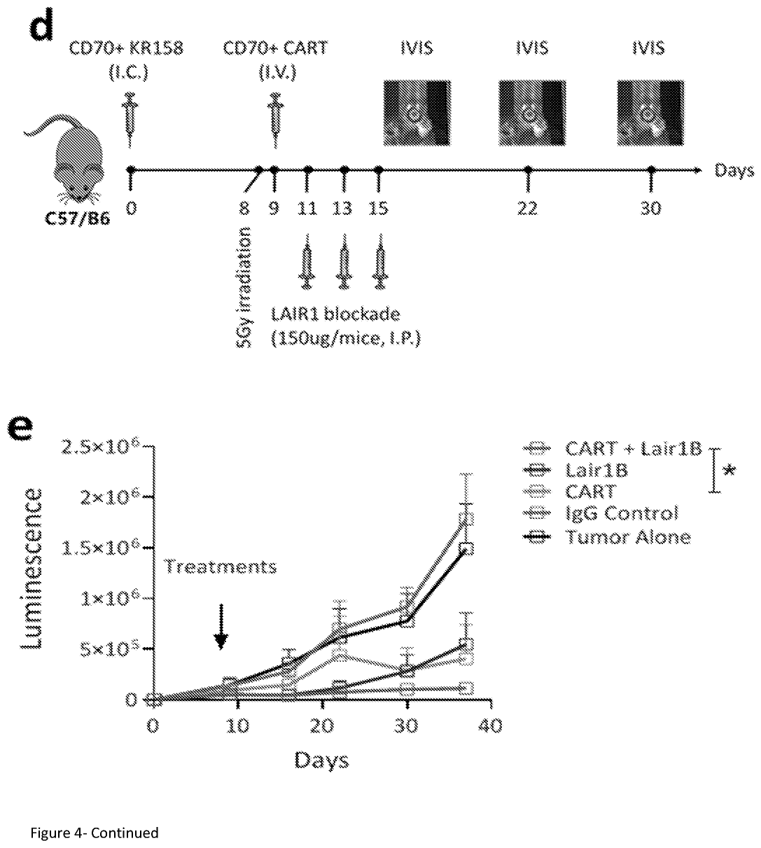

C57BL/6 mice were inoculated with murine glioma tumor, KR-158B, after the tumor was established, the treatment (Isotype IgG, LAIR1, or PD-1 blocking antibodies) was given (i.p. 150 μg/mouse) to the mice, respectively. The LAIR1 blockade prolonged the survival of the tumor burden mice (FIG. 4B). FIG. 4B also shows that LAIR1 blockade also provided a superior antitumor efficacy than the PD-1 blockade.

Results also showed that treatment with an anti-LAIR1 antibody provided a synergistic antitumor response with CAR T cell therapy (see FIGS. 4D and 4E). C57BL/6 mice were inoculated with KR70-Luc GBM (day 0, ˜70% CD70+), and 8 days later, the mice were confirmed tumor formation by IVIS, the TBI (5Gy) was performed and randomly divided into 4 groups (n=5˜7/group). The CD70CAR T cells were given (2×10⁷/mouse, i.v.) to the indicated groups 9 days post tumor inoculation. Two days later, the anti-LAIR1 or IgG control antibody was administered (i.p. 150 μg/mouse) to the treatment group every other day for 3 doses (Days 11, 13, and 15). The tumor size was measured by lVIS.

What is claimed is:

1. A method of treating cancer, comprising administering an anti-LAIR1 antibody to a subject suffering from a cancer that is resistant to checkpoint inhibitor therapy.

2. The method of claim 1, wherein the checkpoint inhibitor therapy is anti PD-1 therapy.

3. The method of claim 2, wherein the anti-PD-1 therapy is pidilizumab, nivolumab or pembrolizumab.

4. The method of any one of claims 1-4, wherein the cancer is glioblastoma.

5. The method of any one of claims 1-5, wherein the antibody is a humanized antibody.

6. The method of any one of claims 1-5, wherein the method further comprises administering a CD70 CAR T cell therapy to the subject.

7. The method of any one of claims 1-6, wherein the antibody binds to an epitope of human LAIR1 within amino acids 22-165 of SEQ ID NO: 1.

8. The method of any one of claims 1-7, wherein the antibody comprises the CDRs set forth in SEQ ID NOs: 10-15.

9. The method of any one or claims 1-7, wherein the antibody comprises the CDRs set forth in SEQ ID NO: 16-21.

10. The method of any one of claims 1-9, further comprising administering a CAR T cell therapy to the subject.
